(19) United States
(12) Patent Application Publication
(10) Pub. No.: US 2019/0194157 A1
(43) Pub. Date: Jun. 27, 2019

(54) COALESCING AGENT DERIVED FROM DIOXOLANE DERIVATIVES

(71) Applicant: PTT GLOBAL CHEMICAL PUBLIC COMPANY LIMITED, Bangkok (TH)

(72) Inventors: SOPHON KAEOTHIP, BANGKOK (TH); PAPAPIDA PORNSURIYASAK, BANGKOK (TH); VORAPONG PIMOLSIRIPHOL, BANGKOK (TH)

(73) Assignee: PTT GLOBAL CHEMICAL PUBLIC COMPANY LIMITED, Bangkok (TH)

(21) Appl. No.: 16/313,568

(22) PCT Filed: Jun. 8, 2017
(86) PCT No.: PCT/TH17/00047

§ 371 Date: Dec. 27, 2018

(30) Foreign Application Priority Data
Jun. 30, 2016 (TH) 1601003922

(51) Int. Cl. C07D 317/24; C07D 407/12; C09D 7/63

(52) U.S. Cl. CPC C07D 317/24; C09D 7/63; C07D 407/12

(57) ABSTRACT

The present invention relates to a coalescing agent as represented in structure (I); wherein; n is integer from 1 to 8; R¹ and R² independently represent group selected from hydrogen atom, alkyl, alkenyl, alkynyl, phenyl, benzyl groups, or optionally cyclic hydrocarbon containing heteroatom; and Y represents group selected from alkyl, alkenyl, alkynyl, phenyl, benzyl groups, or cyclic hydrocarbon containing heteroatom. The said coalescing agent can be used in coating application with efficacy to provide smooth consistent film with chemical and scratch resistant and has no pungent odour, wherein the preparation method of this compound is simplify and employs less harmful chemicals.

### TECHNICAL FIELD

The present invention relates to the field of chemistry, in particular, a coalescing agent and compositions of dioxolane derivatives and the use of dioxolane derivatives as the coalescing agent in coating works.

### BACKGROUND ART

A coating agent is used for the objective for coating of workpiece for decorating including protecting corrosive of the workpiece. This coating agent has been applied in many industrials including, but not limited to automotive industry, interior and exterior construction, and for coating of many other applications. At present, the coating industry has been developed rapidly in order to fulfill the needs and human activities, especially the coating industry for constructing decorations.

However, from the concerns in environmental, health of workers, and current regulatory on the volatile organic compounds (VOCs), there have been many researches and developments in the coating industry because most of the coating compositions are organic solvents. Therefore, at present there have been changes of coating system into water-based formulation or high-solids formulation. Nevertheless, the coalescing agent in water-based paint still has VOC problem as the coalescing agent is the volatile organic compound. The coalescing agent helps in binding of small polymer particles that disperse in water as latex form and become thin film by reducing minimum film forming temperature (MFFT) of latex.

After film has been formed, the coalescing agent moves up to the surface which causes the releasing of VOC to the atmosphere. However, the use of coalescing agent in aqueous coating formulation is still necessary because of its benefit in the property of the obtained coating agent.

Conventional aqueous coating agent has 0.1-10% coalescing agent selected from ester alcohol, ether alcohol, or ester ketone. Said coalescing agent provides uniformly film forming, but has strong odour which is difficult to use. Therefore, there have been researches and developments to produce coalescing agent with low VOC, non-strong odour, and environmental friendly.

Patent document U.S. Pat. No. 4,265,797 disclosed the use of short chain monoalkyl ethers of ethylene or propylene glycol such as propylene glycol methyl ether as the coalescing agent.

Other coalescing agents had been disclosed. For example, ethylene glycol monobutyl ether acetate (Butyl Cellosolve™) had been disclosed in U.S. Pat. No. 3,700,726. Said ester mixture had also been disclosed in U.S. Pat. No. 3,580,876 as the coalescing agent. Moreover, 2,2,4-trimethylpentanediol-1,3-monoisobutyrate (Texanol™) had been disclosed in U.S. Pat. No. 3,312,652.

Patent document U.S. Pat. No. 3,399,158 disclosed the synthesis of dicarboxylic acid diester derivatives with 2 to 6 carbon atoms such as dimethyl succinate, diethyl succinate, and diisopropyl succinate from reaction between carboxylic acid and alcohol. Said dicarboxylic acid diester derivative was used as coalescing agent. The use of diester mixture as the coalescing agent was also disclosed in patent publication WO 2009099948 (A2). Moreover, US 20140243446 A1 disclosed the production of diester derivatives as the coalescing agent from natural precursors such as corn and lignocellulosic cassava using organic acids from biological process to react with alcohol. EP 0026982 A1 disclosed coating formulation from short chain hydrocarbons monoester and diester for the use as the coalescing agent.

Patent document U.S. Pat. No. 8,906,994 disclosed the use of dioxolane derivatives which were 2,2-dimethyl-1,3-dioxolane-4-methanol, solketal, and 2,2-diisobutyl-1,3-dioxolane-4-methanol, solketal as the coalescing agent in paint and waxy coating agent. Patent publication document US 2015361281 A1 disclosed the use of dioxolane ester derivatives from transesterification reaction between dioxolane alcohol and dicarboxylic acid ester which however could not produce effective coalescing agent.

Therefore, the present invention provides derivatives of dioxolane ester as the coalescing agent for aqueous coating formulation that is effective in forming smooth film with chemical and scratch resistant with no strong odour, wherein the preparation process of this compound is not complicate and reducing the use of harmful chemical.

### SUMMARY OF INVENTION

The present invention relates to the coalescing agent derived from dioxolane derivatives as represented in structure (I);

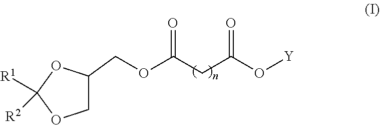

wherein;

n is integer from 1 to 8;

R¹ and R² independently represent group selected from hydrogen atom, alkyl, alkenyl, alkynyl, phenyl, benzyl groups, or optionally cyclic hydrocarbon containing heteroatom; and

Y represents group selected from alkyl, alkenyl, alkynyl, phenyl, benzyl groups, or cyclic hydrocarbon containing heteroatom.

### DETAIL DESCRIPTION OF THE INVENTION

The present invention provides the dioxolane ester derivatives as the coalescing agent for aqueous coating formulation that is effective in forming smooth film with chemical and scratch resistant with no strong odour, wherein the preparation process of this compound is not complicate and and reducing the use of harmful chemical which can be described according to the following embodiments.

Any aspects shown herein also includes the application to other aspects of this invention unless stated otherwise.

### Definitions

Technical terms or scientific terms used here have definitions as by person skilled in the art unless stated otherwise.

Any tools, equipment, methods, or chemicals named here mean tools, equipment, methods, or chemicals being used commonly by person skilled in the art unless stated otherwise that they are tools, equipment, methods, or chemicals specific only in this invention.

Use of singular noun or singular pronoun with “comprising” in claims or specification means “one” and including “one or more”, “at least one”, and “one or more than one” too.

All compositions and/or methods disclosed and claims in this application aim to cover embodiments from any action, performance, modification, or adjustment without any experiment that significantly different from this invention, and obtain with object with utility and resulted as same as the present embodiment according to person ordinary skilled in the art although without specifically stated in claims. Therefore, substitutable or similar object to the present embodiment, including any minor modification or adjustment that clearly seen by person skilled in the art should be construed as remains in spirit, scope, and concept of invention as appeared in appended claims.

Throughout this application, term “about” means any number that appeared or showed here that could be varied or deviated from any error of equipment, method, or personal using said equipment or method.

The coalescing agent includes the organic substance added into the aqueous coating formulation as latex, wherein the coalescing agent softens the polymer particles in latex until they can be combined into film.

The hetero atoms are atoms of non-carbon elements and said atoms, wherein the hetero atoms include, but not limited to IVA group elements such as silicon, germanium, tin, and lead; VA group elements such as nitrogen, phosphorus, arsenic, antimony, and bismuth; VIA group elements such as oxygen, sulphur, selenium, and tellurium; or VIIA group elements such as fluorine, chlorine, bromine, and iodine.

Hereafter, invention embodiments are shown without any purpose to limit any scope of the invention.

This invention relates to the coalescing agent derived from dioxolane derivatives as represented in structure (I);

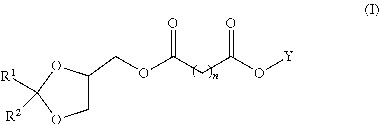

wherein;

n is integer from 1 to 8;

R¹ and R² independently represent group selected from hydrogen atom, alkyl, alkenyl, alkynyl, phenyl, benzyl groups, or optionally cyclic hydrocarbon containing heteroatom; and

Y represents group selected from alkyl, alkenyl, alkynyl, phenyl, benzyl groups, or cyclic hydrocarbon containing heteroatom.

Preferably, n is integer from 1 to 4, and more preferable, n is integer from 2 to 4.

In one embodiment, R¹ and R² are selected from hydrogen atom or alkyl group containing 1 to 8 carbon atoms.

In one embodiment, Y may be selected from alkyl group containing 1 to 4 carbon atoms, preferable, Y may be selected from alkyl group containing 1 to 2 carbon atoms.

In another embodiment, Y may be selected from cyclic hydrocarbon containing heteroatom.

Preferably, Y may be selected from alkyl group containing 1 to 2 carbon atoms or cyclic hydrocarbon containing heteroatom represented in structure (II)

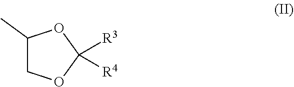

Wherein R³ and R⁴ independently represents group selected from hydrogen atom, alkyl, alkenyl, alkynyl, phenyl, benzyl groups, or optionally cyclic hydrocarbon containing heteroatom. Preferable, R³ and R⁴ are selected from hydrogen atom or alkyl group containing 1 to 8 carbon atoms.

Another objective of the invention is the composition of the coalescing agent from dioxolane derivatives, wherein said composition comprising:

a) 5-60% of coalescing agent with structure (III)

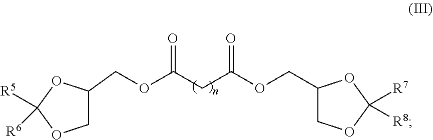

wherein

n is integer from 1 to 8;

R⁵, R⁶, R⁷, and R⁸ independently represent group selected from hydrogen atom, alkyl, alkenyl, alkynyl, phenyl, benzyl groups, or optionally cyclic hydrocarbon containing heteroatom;

b) 40-95% of coalescing agent with structure (IV)

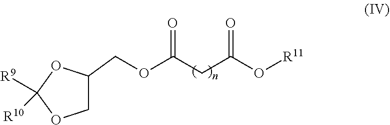

wherein

n is integer from 1 to 8; and

R⁹, R¹⁰, and R¹¹ independently represent group selected from hydrogen atom, alkyl, alkenyl, alkynyl, phenyl, benzyl groups, or optionally cyclic hydrocarbon containing heteroatom.

Preferably, the composition of coalescing agent contains coalescing agent a) in the range of 25-60% and coalescing agent b) in the range of 40-75%.

Preferably, the coalescing agent a), n is integer from 1 to 4, and more preferable, n is integer from 2 to 4.

Preferably, the coalescing agent b), n is integer from 1 to 4, and more preferable, n is integer from 2 to 4.

In one aspect of the invention, the coalescing agent a) R⁵, R⁶, R⁷, and R⁸ are selected from hydrogen atom or alkyl group containing 1 to 8 carbon atoms.

In one aspect of the invention, the coalescing agent b), R⁹ and R¹⁰ are selected from hydrogen atom or alkyl group containing 1 to 8 carbon atoms and R¹¹ is selected from alkyl group containing 1 to 4 carbon atoms. Preferably, R¹¹ is selected from alkyl group containing 1 to 2 carbon atoms.

In one aspect of the invention, the coalescing agent and the composition of the coalescing agent from dioxolane derivatives according to said structure (I), (III), or (IV) can be prepared from process comprising the step of: i) mixing of glycerol and aldehyde or ketone compound at ratio of 1:1 to 1:6 using acid as catalyst; and ii) mixing of compound in step i) and dicarboxylic acid ester compound at ratio of 1:1 to 1:3 for the coalescing agent according to structure (I) and at ratio of 1:2.5 of the coalescing agent according to structure (III) and (IV) using base as catalyst.

Wherein, in reaction in step i), aldehyde or ketone compound is selected from formaldehyde, acetaldehyde, propanal, butanal, pentanal, hexanal, heptanal, octanal, 2-methyl-butanal, crotonaldehyde, acrolein, methacrolein, 2-methylpropanal, trans-2-pentenal, 3-methyl-2-butenal, trans-2-methyl-2-butenal, 2,2-dimethylpropanal, 2-methylbutanal, 3-methylbutanal, 3,3-dimethylbutanal, 2-ethylbutanal, 2-methylpentanal, 3-methylpentanal, 4-methylpentanal, 2-ethylpentanal, 4-pentynal, 2-ethylhexanal, trans-2-hexenal, 4-methylenehex-5-enal, (3 Z)-4-methylhexa-3,5-dienal, 4-methylenehex-5-enal, 2-methyl-heptanal, 2-propyl-pentanal, trans-2-heptenal, cis-4-heptenal, benzaldehyde, o-tolualdehyde, m-tolualdehyde, p-tolualdehyde), trans-cinnamaldehyde, 2,4-dimethylbenzaldehyde, 2,5-dimethylbenzaldehyde, 3,5-dimethylbenzaldehyde, 2,6-dimethylbenzaldehyde, acetone, methyl ethyl ketone, 2-pentanone, 2-hexanone, 2-heptanone, 2-octanone, 2-nonanone, 3-pentanone, 3-hexanone, 3-heptanone, 3-octanone, 3-nonanone, 3-decanone, 3-undecanone, 4-heptanone, 4-octanone, 4-nonanone, 4-decanone, 4-undecanone, 4-dodecanone, 5-nonanone, 5-decanone, 5-undecanone, 5-dodecanone, 5-tridecanone, 6-undecanone, 6-dodecanone, 6-tridecanone, 6-tetradecanone, 7-tridecanone, 7-tetradecanone, 7-pentadecanone, 8-pentadecanone, 8-hexadecanone, 9-heptadecanone, cyclobutanone, cyclopentanone, cyclohexanone, cycloheptanone, 3-buten-2-one, 4-penten-2-one, 5-hexen-2-one, 6-hepten-3-one, 7-octen-4-one, 1-decen-5-one, 1-undecen-5-one, 1,8-nonadien-5-one, 2-methyl-7-octen-4-one, or mixture thereof.

Preferably, the aldehyde or ketone compound is selected from 2-ethyl hexanal, acetone, methyl ethyl ketone, or mixture thereof.

The dicarboxylic acid ester compound for using in reaction in step 2 is selected from dimethylmalonate, diethylmalonate, dipropylmalonate, 2-propenyl-1-[1,3-dioxo-3-(2-propenyloxy)propoxy], dimethylsuccinate, diethylsuccinate, dipropylsuccinate, dibutylsuccinate, dimethylglutarate, diethylglutarate, dipropylglutarate, dibutylglutarate, dimethyladipate, diethyladipate, dipropyladipate, dibutyladipate, or mixture thereof.

Preferably, the dicarboxylic acid ester compound is selected from dimethylsuccinate, diethylsuccinate, diethyladipate, or mixture thereof.

In one aspect of the invention, the coalescing agent according to structure (I) and the composition of the coalescing agent from dioxolane derivatives according to said structure (III) or (IV) can be used in coating composition which comprising:

a) the coalescing agent according to structure (I) or the composition of the coalescing agent from dioxolane derivatives according to said structure (III) or (IV);

b) binder; and

c) solvent;

wherein embodiments and preferred aspects of the coalescing agent according to structure (I) or the composition of the coalescing agent from dioxolane derivatives according to said structure (III) or (IV) in a) are as described above.

In one embodiment, the quantity of the coalescing agent a) is between 1-20% by weight of coating. Preferable is between 3-10% by weight.

The binder in b) may be selected from, but not limited to groups containing alkyd, acrylic, vinyl-acrylic, vinyl acetate/ethylene, polyurethane, epoxy, styrenic, styrenic-acrylic copolymer, styrene alkene copolymer, derivatives of said compounds, or mixture of said compounds.

Preferably, said binder is selected from groups of acrylic, styrenic-acrylic copolymer, or mixture of said binders.

In one aspect, the quantity of said binders is between 10-70% by weight, preferable is between 45-65% by weight.

In one embodiment, the solvent in c) can be selected from solvent compatible with the coalescing agent, wherein said solvent may be selected from, but not limited to water, alcohol, petroleum distillate, ester, glycol, glycol ether, or mixture thereof.

In one optional aspect, the coating composition may further comprising of additives selected from pigment, pigment extender, colorant, surfactant, rheology modifier, texturing agent, defoamer, biocide, wetting agent, dispersing agent, crosslinker, thickener, antifreezing agent, stabilizer, or mixture thereof.

In another optional aspect, the coating composition may have further coalescing agent, wherein the group of additional coalescing agent may be selected from benzoic acid alkyl ester, ester-alcohol, glycol-ether, long chain aliphatic alcohol, aromatic alcohol, or mixture thereof.

The following part is for the demonstrating purpose only, not for the limitation of this invention in any way.

### Example 1: The Preparation of Dioxolane Alcohol Derivatives

Dioxolane Alcohol 1

One hundred grams of glycerol (1.08 moles) and 315.34 g of acetone (5.43 moles) were added into round bottom flask. Then, 1.06 g of sulfuric acid (0.01 mole) was added and refluxed at the temperature of 22-25° C. for 1 hour. Then, mixture was neutralized by 2 N potassium hydroxide dissolved in methanol. Some parts of acetone and water in obtained mixture were evaporated under vacuum. The obtained mixture was purified by distillation process under vacuum condition. The obtained sample from distillation was analyzed by proton-nuclear magnetic resonance spectroscopy (′H-NMR) technique.

Dioxolane Alcohol 2

One hundred grams of glycerol (1.08 moles) and 391.5 g of methyl ethyl ketone (5.43 moles) were added into round bottom flask. Then, 2.07 g of p-toluene sulfuric acid (0.01 mole) was added and refluxed at the temperature of 100° C. for 2 hours and cooled down at room temperature. The mixture was neutralized by 2 N potassium hydroxide dissolved in methanol. Some parts of methyl ethyl ketone and water in obtained mixture were evaporated under vacuum. The obtained mixture was purified by distillation process under vacuum condition. The obtained sample from distillation was analyzed by ¹H-NMR.

Dioxolane Alcohol 3

One hundred grams of glycerol (1.08 moles) and 111.90 g of cyclohexanone (1.14 moles) were added into round bottom flask. Then, 2.06 g of p-toluene sulfuric acid (0.01 mole) was added and refluxed at the temperature of 120° C. for 6 hours and cooled down at room temperature. The mixture was neutralized by 2 N potassium hydroxide dissolved in methanol. Some parts of cyclohexanone and water in obtained mixture were evaporated under vacuum. The obtained mixture was purified by distillation process under vacuum condition. The obtained sample from distillation was analyzed by ¹H-NMR.

Dioxolane Alcohol 4

Fifty grams of glycerol (0.54 mole) and 104.4 g of 2-ethyl hexanal (0.81 mole) were added into round bottom flask that contained 200 mL of acetonitrile. Then, 2.06 g of p-toluene sulfuric acid (10.8 moles) was added and refluxed at the temperature of 60° C. for 4 hours and cooled down at room temperature. Acetonitrile, aldehyde, and water in obtained mixture were evaporated under vacuum. Then, obtained mixture was purified by distillation process under vacuum condition. The obtained sample from distillation was analyzed by ¹H-NMR.

### Example 2: The Preparation of Derivatives of Dioxolane Alcohol 1

Dioxolane Alcohol Ester 1 (DOE 01)

Twenty grams of dimethyl succinate (136.8 mmole) and 36.17 g of Dioxolane alcohol 1 (273.7 mmole) were added in rotund bottom flask. Then, 0.59 g of sodium methoxide (10.9 mmole) was added and refluxed at the temperature of 70° C. for 90 minutes under vacuum condition. Then, the reaction was continued at the temperature of 120° C. under vacuum condition for 30 minutes. Then, the obtained mixture was cooled down at room temperature. After that, the diethyl ether was added and extracted with water. The obtained organic solvent layer was distilled under vacuum condition at the temperature about 40° C. The obtained sample was analyzed by ¹H-NMR and gas chromatography (GC).

Dioxolane Alcohol Ester 2 (DOE 02)

Twenty grams of dimethyl succinate (136.8 mmole) and 54.2 g of Dioxolane alcohol 1 (410.5 mmole) were added into round bottom flask. Then, 0.59 g of sodium methoxide (10.9 mmole) was added and refluxed at the temperature of 90° C. for 90 minutes under vacuum condition. Then, the reaction was continued at the temperature of 120° C. under vacuum condition for 30 minutes. Then, the obtained mixture was cooled down at room temperature. After that, the diethyl ether was added and extracted with water. The obtained organic solvent layer was distilled under vacuum condition at the temperature about 40° C. The obtained sample was analyzed by ¹H-NMR and GC.

Dioxolane Alcohol Ester 3 (DOE 03)

Twenty grams of diethyl adipate (98.8 mmole) and 26.14 g of Dioxolane alcohol 1 (197.8 mmole) were added into round bottom flask. Then, 0.43 g of sodium methoxide (7.9 mmole) was added and refluxed at the temperature of 70° C. for 90 minutes under vacuum condition. Then, the reaction was continued at the temperature of 120° C. under vacuum condition for 30 minutes. Then, the obtained mixture was cooled down at room temperature. After that, the diethyl ether was added and extracted with water. The obtained organic solvent layer was distilled under vacuum condition at the temperature about 40° C. The obtained sample was analyzed by ¹H-NMR and GC.

Dioxolane Alcohol Ester 4 (DOE 04)

Twenty grams of dimethyl malonate (151.4 mmole) and 40 g of Dioxolane alcohol 1 (302.8 mmole) were added into round bottom flask. Then, 0.65 g of sodium methoxide (12.1 mmole) was added and refluxed at the temperature of 70° C. for 90 minutes under vacuum condition. Then, the reaction was continued at the temperature of 120° C. under vacuum condition for 30 minutes. Then, the obtained mixture was cooled down at room temperature. After that, the diethyl ether was added and extracted with water. The obtained organic solvent layer was distilled under vacuum condition at the temperature about 40° C. The obtained sample was analyzed by ¹H-NMR and GC.

### Example 3: The Preparation of Derivatives of Dioxolane Alcohol 2

Dioxolane Alcohol Ester 5 (DOE 05)

Twenty grams of dimethyl succinate (136.8 mmole) and 39.9 g of Dioxolane alcohol 2 (273.7 mmole) were added into round bottom flask. Then, 0.59 g of sodium methoxide (10.9 mmole) was added and reacted at the temperature of 70° C. for 90 minutes under vacuum condition. Then, the reaction was continued at the temperature of 120° C. under vacuum condition for 30 minutes. Then, the obtained mixture was cooled down at room temperature. After that, the diethyl ether was added and extracted with water. The obtained organic solvent layer was distilled under vacuum condition at the temperature about 40° C. The obtained sample was analyzed by ¹H-NMR and GC.

Dioxolane Alcohol Ester 6 (DOE 06)

Ten grams of diethyl adipate (49.4 mmole) and 14.45 g of Dioxolane alcohol 2 (98.8 mmole) were added into round bottom flask. Then, 0.21 g of sodium methoxide (3.9 mmole) was added and reacted at the temperature of 70° C. for 120 minutes under vacuum condition. Then, the reaction was continued at the temperature of 120° C. under vacuum condition for 30 minutes. Then, the obtained mixture was cooled down at room temperature. After that, the diethyl ether was added and extracted with water. The obtained organic solvent layer was distilled under vacuum condition at the temperature about 40° C. The obtained sample was analyzed by ¹H-NMR and GC.

Dioxolane Alcohol Ester 7 (DOE 07)

Ten grams of dimethyl malonate (57.4 mmole) and 16.78 g of Dioxolane alcohol 2 (114.8 mmole) were added into round bottom flask. Then, 0.25 g of sodium methoxide (4.59 mmole) was added and reacted at the temperature of 70° C. for 120 minutes under vacuum condition. Then, the reaction was continued at the temperature of 120° C. under vacuum condition for 30 minutes. Then, the obtained mixture was cooled down at room temperature. After that, the diethyl ether was added and extracted with water. The obtained organic solvent layer was distilled under vacuum condition at the temperature about 40° C. The obtained sample was analyzed by ¹H-NMR and GC.

Dioxolane Alcohol Ester 8 (DOE 08)

Twenty grams of diethyl succinate (114.8 mmole) and 33.55 g of Dioxolane alcohol 2 (229.6 mmole) were added into round bottom flask. Then, 0.49 g of sodium methoxide (9.2 mmole) was added and refluxed at the temperature of 70° C. for 1 hour 30 minutes under vacuum condition. Then, the reaction was continued at the temperature of 120° C. under vacuum condition for 30 minutes. Then, the obtained mixture was cooled down at room temperature. After that, the diethyl ether was added and extracted with water. The obtained organic solvent layer was distilled under vacuum condition at the temperature about 40 DC. The obtained sample was analyzed by 1H-NMR and GC.

### Example 4: The Preparation of Derivatives of Dioxolane Alcohol 3

Dioxolane Alcohol Ester 9 (DOE 09)

Twenty grams of dimethyl succinate (136.8 mmole) and 70.7 g of Dioxolane alcohol 3 (410.5 mmole) were added into round bottom flask. Then, 0.59 g of sodium methoxide (10.9 mmole) was added and refluxed at the temperature of 90° C. for 90 minutes under vacuum condition. Then, the reaction was continued at the temperature of 130° C. under vacuum condition for 30 minutes. Then, the obtained mixture was cooled down at room temperature. After that, the diethyl ether was added and extracted with water. The obtained organic solvent layer was distilled under vacuum condition at the temperature about 40° C. The obtained sample was analyzed by ¹H-NMR and GC.

Dioxolane Alcohol Ester 10 (DOE 10)

Ten grams of dimethyl malonate (75.6 mmole) and 26.0 g of Dioxolane alcohol 3 (151.4 mmole) were measured into round bottom flask. Then, 0.59 g of sodium methoxide (10.9 mmole) was added and refluxed at the temperature of 70° C. for 90 minutes under vacuum condition. Then, the reaction was continued at the temperature of 130° C. under vacuum condition for 30 minutes. Then, the obtained mixture was cooled down at room temperature. After that, the diethyl ether was added and extracted with water. The obtained organic solvent layer was distilled under vacuum condition at the temperature about 40° C. The obtained sample was analyzed by ¹H-NMR and GC.

### Example 5: The Preparation of Derivatives of Dioxolane Alcohol 4

Dioxolane Alcohol Ester 11 (DOE 11)

Twenty grams of dimethyl succinate (136.8 mmole) and 83.0 g of Dioxolane alcohol 4 (410.5 mmole) were added into round bottom flask. Then, 0.59 g of sodium methoxide (10.9 mmole) was added and refluxed at the temperature of 70° C. for 90 minutes under vacuum condition. Then, the reaction was continued at the temperature of 150° C. under vacuum condition for 30 minutes. Then, the obtained mixture was cooled down at room temperature. After that, the diethyl ether was added and extracted with water. The obtained organic solvent layer was distilled under vacuum condition at the temperature about 40° C. The obtained sample was analyzed by ¹H-NMR and GC.

Dioxolane Alcohol Ester 12 (DOE 12)

Ten grams of diethyl succinate (57.4 mmole) and 34.8 g of Dioxolane alcohol 4 (172.2 mmole) were added into round bottom flask. Then, 0.25 g of sodium methoxide (4.5 mmole) was added and refluxed at the temperature of 90° C. for 90 minutes under vacuum condition. Then, the reaction was continued at the temperature of 150° C. under vacuum condition for 30 minutes. Then, the obtained mixture was cooled down at room temperature. After that, the diethyl ether was added and extracted with water. The obtained organic solvent layer was distilled under vacuum condition at the temperature about 40° C. The obtained sample was analyzed by ¹H-NMR and GC.

### Example 6: Properties Testing of Dioxolane Ester Derivatives According to the Invention

Said dioxolane ester derivatives can be analyzed by methods described in the following.

Film Forming Efficacy Testing

Film forming efficacy testing of the coalescing agent DOE 01 to DOE 12 comparing to the commercial coalescing agent Texanol™ and Butyl Cellosolve™ could be done according to the following steps.

1. Twenty grams of acrylic or styrenic-acrylic copolymers binder were added into testing beaker.

2. Coalescing agent at each concentration from 1 to 15% wt of binder was slowly added into binder from (1). Each obtained mixture was stirred until homogenous. This took 10-15 minutes. All prepared samples (1-15%) were done.

3. Films obtained from binders and coalescing agents in step 2 were drawn onto supporting paper using wire coater until 100 micron thick film layers were obtained. Then, the obtained films were left to be dried at room temperature.

4. When films were dried, the appearances of films were observed. The obtained films must be transparent and had no cracking. The percentages of the coalescing agents used to produce good films were recorded.

Testing of Drying Property of the Coalescing Agents

The testing of drying property of the coalescing agents DOE 01 to DOE 12 comparing to the commercial coalescing agent Texanol™ and Butyl Cellosolve™ could be done according to the following steps.

1. Twenty grams of acrylic or styrenic-acrylic copolymers binder were added into testing beaker, and the coalescing agents DOE 01 to DOE 12 which is at the capable concentration to produce the best film were added. Each coalescing agents and binders were mixed together and stirred until homogeneously mixed.

2. Films of each coalescing agents and the binders obtained from step 1 were drawn onto supporting paper using wire coater until 100 micron thick film layers were obtained. Then, the obtained films were left to be dried at room temperature.

3. The prepared films were touch by finger. The time that there was no fingerprint shown on the film was recorded.

Odour Testing of the Coalescing Agents

The odour testing of the coalescing agents DOE 01 to DOE 12 comparing to the commercial coalescing agent Texanol™ and Butyl Cellosolve™ can be performed according to the following steps.

1. Ten grams of the coalescing agents DOE 01 to DOE 12 were added into each testing beaker.

2. Five assessors were asked to smell and recorded the odour intensity from 0-3, wherein 3 meant highest odour intensity and 0 meant no odour detected.

VOC Testing

The testing of VOC of the coalescing agents DOE 01 to DOE 12 comparing to the commercial coalescing agent Texanol™ and Butyl Cellosolve™ could be performed according to the ISO 11890-2 standard.

Composition of the Coalescing Agents According to the Invention

The composition of the synthetic coalescing agents DOE 01 to DOE 12 had compositions as shown in table 1. It was found that the coalescing agents DOE 01, DOE 03, DOE 05, DOE 06, DOE 07, and DOE 08 were mixtures between monodioxolane diester and bis-dioxolane diester in which had monodioxolane diester in the range of 44-81% and bis-dioxolane diester in the range of 19-56%. The coalescing agents DOE 02, DOE 09, and DOE 10 were bis-dioxolane ester, and the coalescing agents DOE 11 and DOE 12 were monodioxolane diester.

The Properties of the Coalescing Agents According to the Invention

The properties of the coalescing agents DOE 01 to DOE 12 comparing to the commercial coalescing agent Texanol™ and Butyl Cellosolve™ were shown in table 2. It was found that the coalescing agents DOE 01, DOE 02, DOE 03, DOE 05, DOE 08, and DOE 11 according to the invention had same level of the film forming efficacy as those commercial coalescing agents. That means the coalescing agents according to the invention were added only in low amounts in order to form polymer films as same as the commercial coalescing agents. Moreover, the coalescing agents according to the invention needed short drying time as same as the commercial coalescing agents. For the odour and VOC value, the coalescing agents according to the invention gave less intensity for odour than the commercial coalescing agents. This showed that the coalescing agents according to the invention had similar efficacy as those commercial coalescing agents with lower VOC value.

TABLE 1
Compositions of the coalescing agents DOE 01 to DOE 12

| Sample | Structure (% by mass) | Structure (% by mass) |
|---|---|---|
| DOE 01 | 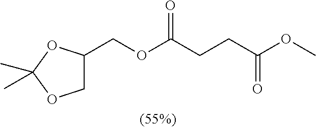 | 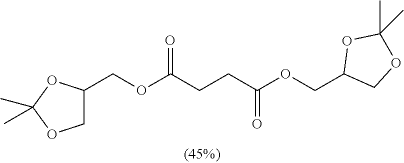 |
| DOE 02 | — | 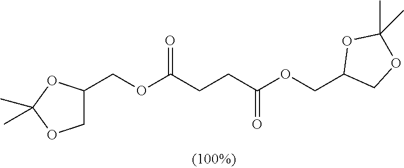 |
| DOE 03 | 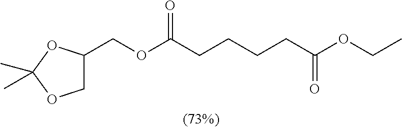 [SMILES: CCOC(=O)CCCCC(=O)OCC1COC(C)(C)O1] | 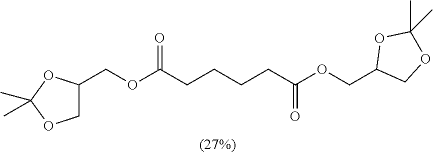 |
| DOE 04 | 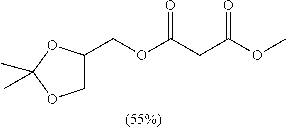 | 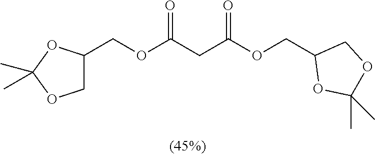 |
| DOE 05 | 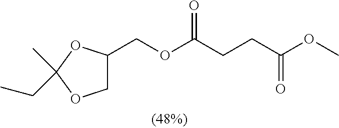 [SMILES: CCC1(C)OCC(COC(=O)CCC(=O)OC)O1] | 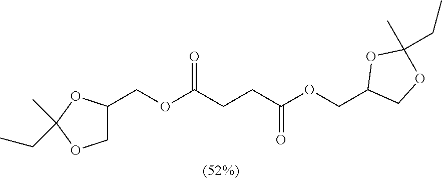 [SMILES: CCC1(C)OCC(COC(=O)CCC(=O)OCC2COC(C)(CC)O2)O1] |
| DOE 06 | 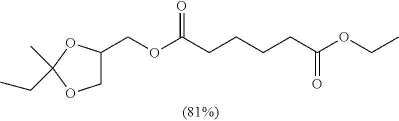 [SMILES: CCOC(=O)CCCCC(=O)OCC1COC(C)(CC)O1] | 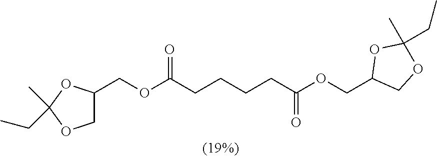 [SMILES: CCC1(C)OCC(COC(=O)CCCCC(=O)OCC2COC(C)(CC)O2)O1] |

TABLE 2
Properties of the coalescing agents according to the invention comparing to the commercial coalescing agents

| | Acrylic Binder | | | | Styrenic-Acrylic Copolymers Binder | | | | |
|---|---|---|---|---|---|---|---|---|---|
| | Amount used | | Drying | | Amount used to | | Drying | | |
| | to form film | Film | of film | | form film | Film | of film | | VOC |
| Sample | (% by weight) | characteristic | (min) | Odour | (% by weight) | characteristic | (min) | Odour | (g/l) |
| Texanol ™ | 5 | transparent | 28 | 3 | 5 | transparent | 20 | 3 | 4.48 |
| Butyl | 9 | transparent | 25 | 3 | 6 | transparent | 20 | 3 | 4.98 |
| Cellosolve ™ | | | | | | | | | |
| DOE 01 | 6 | transparent | 26 | 1 | 5 | transparent | 22 | 1 | 1.21 |
| DOE 02 | 6 | transparent | 27 | 0 | 5 | transparent | 19 | 0 | 1.15 |
| DOE 03 | 5 | transparent | 28 | 1 | 6 | transparent | 19 | 1 | 1.13 |
| DOE 04 | film could not | — | — | — | film could not | — | — | — | — |
| | be formed | | | | be formed | | | | |
| DOE 05 | 8 | transparent | 27 | 1 | 6 | transparent | 18 | 1 | 1.27 |
| DOE 06 | film could not | — | — | — | film could not | — | — | — | — |
| | be formed | | | | be formed | | | | |
| DOE 07 | film could not | — | — | — | film could not | — | — | — | — |
| | be formed | | | | be formed | | | | |
| DOE 08 | 6 | transparent | 22 | 1 | 6 | transparent | 17 | 1 | 1.28 |
| DOE 09 | film could not | — | — | — | film could not | — | — | — | — |
| | be formed | | | | be formed | | | | |
| DOE 10 | film could not | — | — | — | film could not | — | — | — | — |
| | be formed | | | | be formed | | | | |
| DOE 11 | 5 | transparent | 26 | 1 | 5 | transparent | 20 | 1 | 1.22 |
| DOE 12 | film could not | — | — | — | film could not | — | — | — | — |
| | be formed | | | | be formed | | | | |

### BEST MODE OR PREFERRED EMBODIMENT OF THE INVENTION

Best mode or preferred embodiment of the invention is as provided in the detail description of the invention.

1.-30. (canceled)

31. A coalescing agent derived from dioxolane derivatives as represented in structure (I); 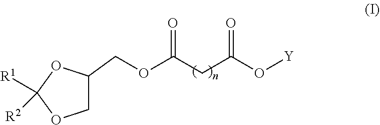
wherein;
n is integer from 1 to 8;
R¹ and R² independently represent group selected from hydrogen atom, alkyl, alkenyl, alkynyl, phenyl, benzyl groups, or optionally cyclic hydrocarbon containing heteroatom; and
Y represents group selected from alkyl, alkenyl, alkynyl, phenyl, benzyl groups, or cyclic hydrocarbon containing heteroatom.

32. The coalescing agent derived from dioxolane derivatives according to claim 31, wherein n is integer from 1 to 4.

33. The coalescing agent derived from dioxolane derivatives according to claim 31, wherein R¹ and R² are selected from hydrogen atom or alkyl group containing 1 to 8 carbon atoms, Y is selected from alkyl group containing 1 to 4 carbon atoms and cyclic hydrocarbon containing heteroatom.

34. The coalescing agent derived from dioxolane derivatives according to claim 31, wherein Y is selected from cyclic hydrocarbon containing heteroatom represented in structure (II) 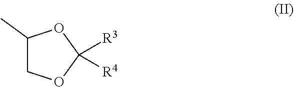
wherein R³ and R⁴ independently represents group selected from hydrogen atom, alkyl, alkenyl, alkynyl, phenyl, benzyl groups, or optionally cyclic hydrocarbon containing heteroatom.

35. The coalescing agent derived from dioxolane derivatives according to claim 34, wherein R³ and R⁴ are selected from hydrogen atom or alkyl group containing 1 to 8 carbon atoms.

36. The coalescing agent derived from dioxolane derivatives according to claim 31, wherein said coalescing agent is prepared from method comprising steps:
i) mixing glycerol and aldehyde or ketone compound at ratio of 1:1 to 1:6 using acid as catalyst;
ii) mixing the compound obtained from step i) and dicarboxylic acid ester compound at ratio of 1:1 to 1:3 using base as catalyst;

37. The coalescing agent derived from dioxolane derivatives according to claim 36, wherein the aldehyde or ketone compound in the reaction in step i) is selected from 2-ethyl hexanal, acetone, methyl ethyl ketone, or mixture thereof.

38. The coalescing agent derived from dioxolane derivatives according to claim 36, wherein the dicarboxylic acid ester compound in reaction in step ii) is selected from dimethylsuccinate, diethylsuccinate, diethyladipate, or mixture thereof.

39. A composition of coalescing agent derived from dioxolane derivatives, wherein said composition comprising:
a) 5-60% of coalescing agent with structure (III) 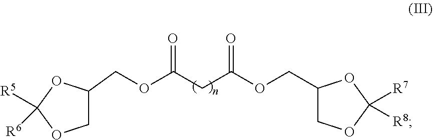
wherein
n is integer from 1 to 8;
R⁵, R⁶, R⁷, and R⁸ independently represent group selected from hydrogen atom, alkyl, alkenyl, alkynyl, phenyl, benzyl groups, or optionally cyclic hydrocarbon containing heteroatom;
b) 40-95% of coalescing agent with structure (IV) 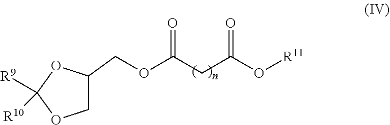
wherein
n is integer from 1 to 8;
R⁹, R¹⁰, and R¹¹ independently represent group selected from hydrogen atom, alkyl, alkenyl, alkynyl, phenyl, benzyl groups, or optionally cyclic hydrocarbon containing heteroatom

40. The composition of coalescing agent derived from dioxolane derivatives according to claim 39, wherein said coalescing agent is blend of coalescing agent derived from dioxolane derivatives containing coalescing agent a) in the range of 25-60% and coalescing agent b) in the range of 40-75%.

41. The composition of coalescing agent derived from dioxolane derivatives according to claim 39, wherein the coalescing agent a), n is integer from 1 to 4 and the coalescing agent b), n is integer from 1 to 4.

42. The composition of coalescing agent derived from dioxolane derivatives according to claim 39, wherein the coalescing agent a), R⁵, R⁶, R⁷, and R⁸ are selected from hydrogen atom or alkyl group containing 1 to 8 carbon atoms and wherein the coalescing agent b), R⁹ and R¹⁰ are selected from hydrogen atom or alkyl group containing 1 to 8 carbon atoms, and R¹¹ is selected from alkyl group containing 1 to 4 carbon atoms.

43. The composition of coalescing agent derived from dioxolane derivatives according to claim 39, wherein said coalescing agent is prepared from method comprising steps:
i) mixing glycerol and aldehyde or ketone compound at ratio of 1:1 to 1:6 using acid as catalyst;
ii) mixing compound obtained from step i) and dicarboxylic acid ester compound at ratio of 1:1 to 1:25 using base as catalyst;

44. A coating composition obtained from coalescing agent derived from dioxolane derivatives according to claim 39.
